(19) United States
(12) Patent Application Publication
(10) Pub. No.: US 2008/0009835 A1
(43) Pub. Date: Jan. 10, 2008

(54) Fluid dispensing apparatus with flow rate control

(76) Inventors: Marshall S. Kriesel, Saint Paul, MN (US); Joshua W. Kriesel, San Francisco, CA (US); Alan D. Langerud, Saint Paul, MN (US)

Correspondence Address: JAMES E. BRUNTON, ESQ., P. O. BOX 29000, GLENDALE, CA, 91209, US

(21) Appl. No.: 11/353,762

(22) Filed: Feb. 13, 2006

Related U.S. Application Data
(60) Provisional application No. 60/654,552, filed on Feb. 17, 2005.

(51) Int. Cl. A61K 9/22

(52) U.S. Cl. 604/890.1; 604/82

(57) ABSTRACT

An apparatus for delivering fluids at precisely controlled rates to ambulatory patients. The invention comprises a housing having a fluid reservoir to contain fluids to be delivered to the patient, a novel stored energy membrane for expelling fluid from the reservoir and a unique flow control assembly in communication with the fluid reservoir for the precise infusion of pharmaceutical fluids to ambulatory patients at precisely controlled rates. The flow control assembly includes a novel rate control member having a plurality of fluidic micro-channels through which the fluid is selectively directed.

### This is a Non-Provisional Application claiming the benefit of co-pending Provisional Application No. 60/654,552 filed Feb. 17, 2005.

### BACKGROUND OF THE INVENTION

1. Field of the Invention

The present invention relates generally to fluid delivery devices. More particularly, the invention concerns an improved apparatus for infusing medicinal agents into an ambulatory patient at specific rates over extended periods of time. The apparatus includes both novel vial assembly fill means for filling the reservoir of the device with medicinal agents and unique flow rate control means for precisely controlling the rate of flow of medicinal agents toward the patient.

2. Discussion of the Invention

Many medicinal agents require an intravenous route for administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by mediating its therapeutic effectiveness. Certain classes of new pharmacologic agents possess a very narrow range of therapeutic effectiveness, for instance, too small a dose results in no effect, while too great a dose results in toxic reaction.

In the past, prolonged infusion of fluids has generally been accomplished using gravity flow methods, which typically involve the use of intravenous administration sets and the familiar bottle suspended above the patient. Such methods are cumbersome, imprecise and require bed confinement of the patient. Periodic monitoring of the apparatus by the nurse or doctor is required to detect malfunctions of the infusion apparatus.

Devices from which liquid is expelled from a relatively thick-walled bladder by internal stresses within the distended bladder are well known in the prior art. Such bladder, or “balloon”-type, devices are described in U.S. Pat. No. 3,469,578, issued to Bierman, and in U.S. Pat. No. 4,318,400, issued to Perry. The devices of the aforementioned patents also disclose the use of fluid flow restrictors external of the bladder for regulating the rate of fluid flow from the bladder.

The prior art bladder-type infusion devices are not without drawbacks. Generally, because of the very nature of the bladder or “balloon” configuration, the devices are unwieldy and are difficult and expensive to manufacture and use. Further, the devices are somewhat unreliable and their fluid discharge rates are frequently imprecise.

The apparatus of the present invention overcomes many of the drawbacks of the prior art by eliminating the bladder and making use of elastomeric films and similar materials, which, in cooperation with a base, define a fluid reservoir that contains the fluid which is to be dispensed. The elastomeric film membrane controllably forces fluid within the reservoir toward the reservoir outlet.

The elastomeric film materials used in the apparatus of the present invention, as well as various alternate constructions of the apparatus, are described in detail in U.S. Pat. No. 5,205,820 issued to one of the present inventors. Therefore, U.S. Pat. No. 5,205,820 is hereby incorporated by reference in its entirety as though fully set forth herein. U.S. Pat. No. 6,086,561 also issued to one of the present inventors describes various alternate constructions and modified physical embodiments of the invention. This latter patent is also hereby incorporated by reference in its entirety as though fully set forth herein.

The apparatus of the present invention can be used with minimal professional assistance in an alternate health care environment, such as the home. By way of example, the apparatus can be used for the continuous infusion of antibiotics, hormones, steroids, blood clotting agents, analgesics, and like medicinal agents. Similarly, the devices can be used for I-V chemotherapy and can accurately deliver fluids to the patient in precisely the correct quantities and at extended microfusion rates over time.

The apparatus of the present invention, which includes a unique vial fill assembly for filling the reservoir of the apparatus, also includes a novel fluid flow rate control assembly for precisely controlling the rate of fluid flow from the apparatus reservoir to the patient. More particularly, the fluid flow rate control assembly comprises a novel flow control plate that is positioned intermediate the apparatus reservoir and the administration set that carries the fluid to the patient. The flow control plate is provided with a plurality of elongated fluidic flow control micro-channels that are in communication with a rate selector member that is rotatably carried by the apparatus housing. Rotation of the rate selector member places a selected one of the flow control micro-channels in communication with the administration set and precisely controls the rate of fluid flow toward the patient.

A number of fluid flow rate control devices for use in controlling the rate of fluid flow from a fluid supply toward a patient have been suggested in the past. Exemplary of such prior art devices are those described in U.S. Pat. No. 6,095,491 issued to one of the present inventors. This patent describes a readily adjustable flow rate control device having a movable flow control member which includes a plurality of spaced-apart flow restrictors which are adapted to be selectively positioned intermediate a fluid flow path extending between a fluid supply line and a fluid delivery line. In one form of the invention the flow restrictors take the form of a plurality of porous rate control frits which can be selectively moved into index with the fluid flow path.

Another prior art fluid flow control device is described in U.S. Pat. No. 5,499,968 issued to Milijasevic et al. This patent describes various constructions of in-line fluid flow controllers which are adapted primarily for use with a conventional fluid administration set of the type used for infusion of fluid into the body of a patient. In one embodiment, the Milijasevic et al., fluid flow controllers comprise a housing, a chamber therein and an inlet to and an outlet from the chamber. The housing is adapted to receive therewithin at least one flow restrictor having an orifice configured to control the rate of fluid flow therethrough and into the body of the patient. In an alternate embodiment, the controller is adapted with a series of fluid passageways which are linked with a series of orifice plates held in position by a wedge.

Another somewhat similar prior art fluid flow rate control device is disclosed in U.S. Pat. No. 4,781,698 issued to Parren. The Parren device comprises a conventional roller clamp which is connected to a drop chamber. The drop chamber controls the size of the droplets flowing toward the roller clamp, and the roller clamp controls the rate of fluid flow through the delivery line. The Parren apparatus includes a disk having a discharge opening which is selectively alignable with one or more drop tubes and includes a flexible edge or wiper means formed around the discharge opening to provide a seal between the disk and the selected drop tube to prevent fluid from seeping between the disk and the mounting plate.

A common drawback of many of the prior art flow controllers is that the controllers are often complex in construction, are difficult and costly to manufacture, are often somewhat unreliable and lack ease of adjustability to quickly and expeditiously vary the rate of fluid through the device. The rate control assembly of the present invention overcomes these drawbacks by providing a highly precise flow rate control assembly which is particularly well-suited for precisely dispensing medicaments to a patient in a home care environment.

### SUMMARY OF THE INVENTION

It is an object of the present invention to provide an apparatus for delivering fluids at a precisely controlled rate which comprises a fluid dispensing component having a fluid reservoir for containing the fluids to be delivered and a reservoir fill component which can be removably interconnected with the fluid dispensing component. More particularly, it is an object of the invention to provide such an apparatus in which the reservoir fill component can be used to controllably fill the reservoir of the dispensing component and in which the dispensing component can be used for the precise infusion of pharmaceutical fluids to an ambulatory patient at precisely controlled rates.

It is another object of the invention to provide an apparatus of the aforementioned character which is highly reliable and easy-to-use by lay persons in a non-hospital environment.

Another object of the invention is to provide an apparatus which can readily be filled in the field shortly prior to use using the novel reservoir fill component which can be removably interconnected to the lower surface of the base of the fluid dispenser.

Another object of the invention is to provide an apparatus of the aforementioned character, which includes a novel fluid flow rate control assembly disposed intermediate the fluid reservoir outlet and the outlet port of the device.

Another object of the invention is to provide an apparatus which includes a fluid flow rate control assembly as described in the preceding paragraph which includes a novel flow control plate that is provided with a plurality of elongated fluidic flow control micro-channels that are in communication with a rate selector member that is rotatably carried by the apparatus housing. Rotation of the rate selector member places a selected one of the flow control micro-channels in communication with the medicament dispenser and in communication with a patient to precisely control the rate of fluid flow toward the patient.

Another object of the invention is to provide an apparatus which includes a novel fluid flow rate control assembly as described in the preceding paragraphs in which the fluidic flow control micro-channels comprise meandering micro-channels of various lengths, depths, widths and configurations.

Another object of the invention is to provide a device of the character described which includes priming means for priming the various fluid passageways of the device and purging the fluid passageways of gases that may be contained therein prior to the delivery of the medicinal fluids to the administration line of the device. More particularly, an object of the invention is to provide such a device which includes a flow control plate that is provided with a priming channel that is in communication with the plurality of elongated fluidic flow control channels formed in a rate control member and is also in communication with the rate selector member that is rotatably carried by the device housing.

Another object of the invention is to provide an apparatus which includes a novel fluid flow rate control assembly of the class described in which the flow rate selector member can be locked against rotation once a particular fluidic flow control channel is selected.

Another object of the invention is to provide a unique fill assembly for use in controllably filling the fluid reservoir of the apparatus.

Another object of the present invention is to provide an apparatus of the aforementioned character in which the fill assembly comprises a vial assembly that can be pre-filled with a wide variety of medicinal fluids.

Another object of the present invention is to provide a fill assembly of the type described in the preceding paragraph in which the pre-filled vial assembly is partially received within the housing of a novel syringe assembly that can be operably interconnected with the housing of the fluid dispensing apparatus using a sterile coupling.

Another object of the invention is to provide a novel fill assembly for use with the fluid dispensing apparatus which is easy to use, is inexpensive to manufacture, and one which maintains the fill assembly in an aseptic condition until time of use.

Other objects of the invention will become more apparent from the discussion which follows.

### BRIEF DESCRIPTION OF THE DRAWINGS

FIG. 1 is a generally perspective view of one form of the fluid delivery apparatus of the invention.

FIG. 2 is a generally perspective view of the forward portion of the apparatus housing shown in FIG. 1 illustrating the administration set storage compartment of the apparatus in an open configuration.

FIG. 3 is an enlarged, longitudinal, cross-sectional view of the fluid delivery apparatus of the invention shown in FIG. 1.

FIG. 4 is a generally perspective, fragmentary, exploded view of a portion of the embodiment of the invention shown in FIG. 1, illustrating the path of fluid flow through the apparatus.

FIG. 5 is a generally perspective, exploded view of the forward portion of the apparatus housing showing the rate control housing exploded away from the administration set storage compartment.

FIG. 6 is a generally perspective, exploded view of the rearward, reservoir defining portion of the apparatus.

FIG. 7 is an enlarged, generally perspective, exploded view of the fluid flow control portion of the apparatus of one form of the invention.

FIG. 8 is a plan view of the rear face of the reservoir housing closure member showing the configuration of the fluid diffusion component of the apparatus of the invention.

FIG. 9 is a cross-sectional view taken along lines 9-9 of FIG. 8.

FIG. 10 is a top plan view of the reservoir closure member shown in FIG. 8.

FIG. 11 is a front view of the reservoir closure member shown in FIG. 8.

FIG. 12 is a cross-sectional view taken along lines 12-12 of FIG. 11.

FIG. 13 is fragmentary, cross-sectional view illustrating the fluid flow path through the fluid diffusion component and into the fluid flow rate control subassembly of the apparatus of the invention.

FIG. 14 is a cross-sectional view taken along lines 14-14 of FIG. 15.

FIG. 15 is a front view of the rate control housing of the apparatus and a front view of a portion of one form of the flow control assembly of the apparatus of the invention.

FIG. 16 is a top plan view of one form of the flow rate control subassembly of the fluid flow control assembly of the apparatus of the invention.

FIG. 17 is front view of the flow rate control subassembly shown in FIG. 16.

FIG. 18 is an enlarged, cross-sectional view taken along lines 18-18 of FIG. 16.

FIG. 19 is a top plan view of the base of the flow rate control subassembly shown in FIG. 17.

FIG. 19A is a fragmentary cross-sectional view of one of the fluidic micro channels of one form of the flow control means of the invention.

FIG. 20 is a front view of the base of the flow rate control subassembly shown in FIG. 19.

FIG. 21 is a left end view of one form of the rate control cylinder of the fluid flow control assembly of the apparatus of the invention.

FIG. 22 is a cross-sectional view taken along lines 22-22 of FIG. 21.

FIG. 23 is a right end view of the rate control cylinder of the fluid flow control assembly of the apparatus of the invention.

FIG. 24 is a cross-sectional view taken along lines 24-24 of FIG. 22.

FIG. 25 is a cross-sectional view taken along lines 25-25 of FIG. 22.

FIG. 26 is a rear view of the rate control knob of the selector means of the apparatus of the invention.

FIG. 27 is a side view of the rate control knob shown in FIG. 26.

FIG. 28 is a front view of the rate control knob shown in FIG. 26.

FIG. 29 is a top plan view of a portion of one form of the fluid flow control assembly of the apparatus of the invention.

FIG. 30 is a cross-sectional view taken along lines 30-30 of FIG. 29.

FIG. 31 is a front view of the portion of the fluid flow control assembly shown in FIG. 29.

FIG. 32 is a cross-sectional view taken along lines 32-32 of FIG. 31.

FIG. 33 is a side view of the portion of the fluid flow control assembly shown in FIG. 29.

FIG. 34 is a bottom view of the portion of the fluid flow control assembly shown in FIG. 29.

FIG. 35 is a fragmentary rear view of one form of the control knob and the locking means of the fluid flow control assembly of the apparatus of the invention.

FIG. 36 is a cross-sectional view taken along lines 36-36 of FIG. 35.

FIG. 37 is a cross-sectional view similar to FIG. 36, but showing the locking means and a locked configuration.

FIG. 38 is a bottom view of the locking means of the invention shown in FIG. 36.

FIG. 39 is a bottom view similar to FIG. 38, but showing the locking means of the invention in an unlocked, retracted configuration.

FIG. 40 is a generally perspective, exploded view of one form of the fill means, or filling syringe of the apparatus of the invention for use in the filling the apparatus reservoir.

FIG. 41 is an exploded, longitudinal cross-sectional view of one form of the filling syringe and cooperating fill vial of the apparatus of the invention.

FIG. 42 is a cross-sectional view similar to FIG. 41, but showing the fill vial mated with the filling syringe.

FIG. 43 is a generally perspective, exploded view of an alternate form of fill means, or filling syringe of the apparatus of the invention.

FIG. 44 is a longitudinal, cross-sectional, exploded view of the filling syringe, cooperating fill vial and pusher means of one form of the fill means of the invention.

FIG. 45 is a longitudinal cross-sectional view, similar to FIG. 44, but showing the components in an assembled configuration.

FIG. 46 is an enlarged, longitudinal, cross-sectional view similar to FIG. 3, but showing the alternate form of fill means, mated with the fluid delivery apparatus of the invention.

FIG. 47 is a generally perspective, exploded view of the forward portion of an alternate form of the apparatus housing of the invention showing the rate control housing exploded away from the rearward, reservoir defining portion of the apparatus.

FIG. 48 is a top plan view of the rate control housing of the apparatus.

FIG. 49 is a front view of the rate control housing of the apparatus.

FIG. 50 is a cross-sectional view taken along lines 50-50 of FIG. 49.

FIG. 51 is a top plan view of an alternate form of the rate control cylinder of the fluid flow control assembly of the apparatus of the invention.

FIG. 52 is a left-end view of the rate control cylinder shown in FIG. 51.

FIG. 53 is a right-end view of the rate control cylinder shown in FIG. 51.

FIG. 54 is a cross-sectional view taken along lines 54-54 of FIG. 51.

FIG. 55 is a top plan view of one form of the flow rate control subassembly of the fluid flow control assembly of the alternate form of the apparatus of the invention.

FIG. 56 is a cross-sectional view taken along lines 56-56 of FIG. 55.

FIG. 57 is an enlarged cross-sectional view taken along lines 57-57 of FIG. 55.

FIG. 58 is a top plan view of the base, or rate control member of the flow rate control subassembly shown in FIG. 55.

FIG. 59 is a side view of an alternate form of flow rate control assembly of the present invention.

FIG. 60 is a top plan view of the flow rate control assembly of the apparatus illustrated in FIG. 59.

FIG. 61 is an enlarged cross-sectional view taken along lines 61-61 of FIG. 60.

FIG. 62 is an enlarged cross-sectional view taken along lines 44-44 of FIG. 60.

FIG. 63 is a top plan view of the cover member of the flow rate control assembly of the apparatus illustrated in FIG. 59.

FIG. 64 is a view taken along lines 64-64 of FIG. 63.

FIG. 65 is a bottom plan view of the cover member of the flow rate control assembly of the apparatus illustrated in FIG. 59.

FIG. 66 is an enlarged view taken along lines 64-64 of FIG. 61.

FIG. 67 is an enlarged view taken along lines 67-67 of FIG. 65.

FIG. 68 is an enlarged view taken along lines 68-68 of FIG. 63.

FIG. 68A is a fragmentary cross-sectional view similar to FIG. 68, but showing the compression of an elastomeric cover port as the rate control assembly is mated with the housing.

FIG. 69 is an enlarged view taken along lines 69-69 of FIG. 65.

FIG. 70 and is a side view of the base member of the flow rate control assembly of this latest form of the invention.

FIG. 71 is a bottom plan view of the base member the flow rate control assembly of this latest form of the invention.

FIG. 72 is a generally tabular view illustrating the fluidic properties of one form of the fluid rate control member, or rate control chip of the form of the flow rate control device shown in FIG. 47.

### DESCRIPTION OF THE INVENTION

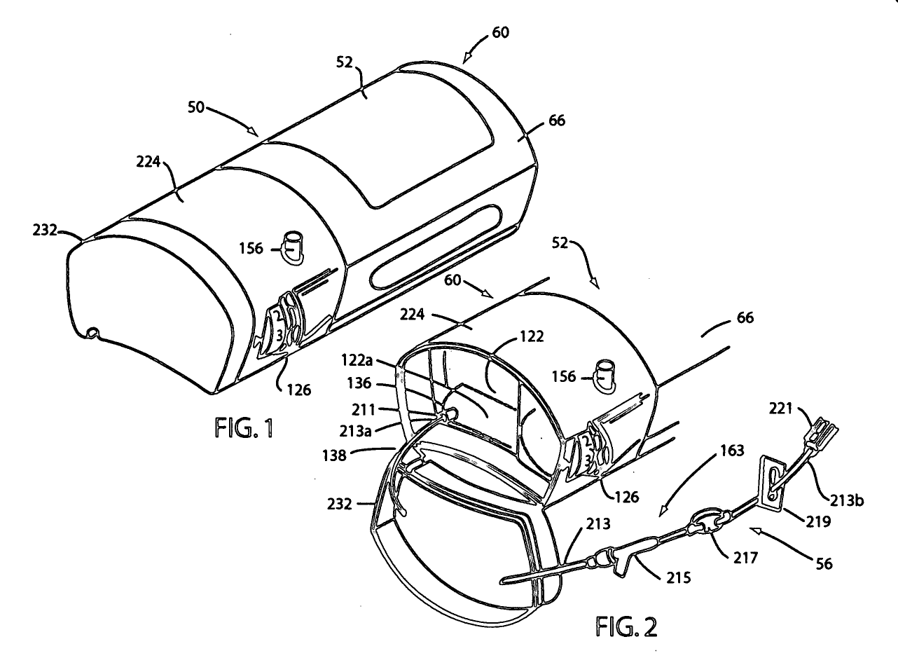

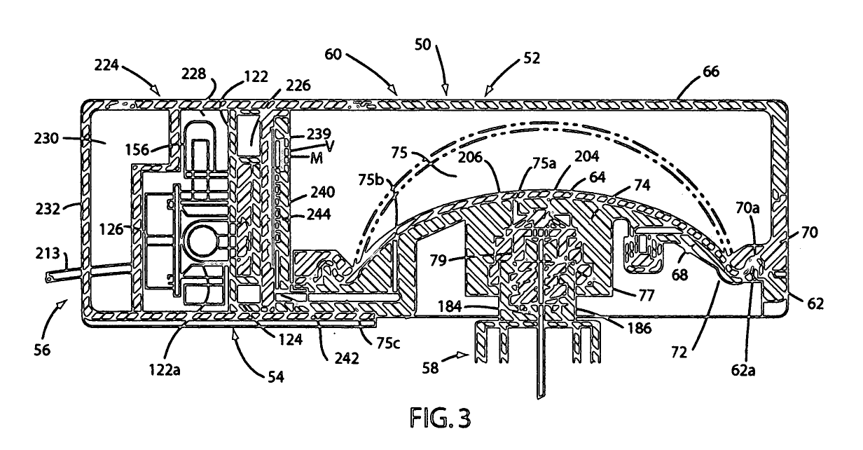

Referring to the drawings and particularly to FIGS. 1 through 4, one form of the fluid dispensing apparatus of the invention is there shown and generally designated by the numeral 50 (see FIG. 1). As best seen in FIG. 3, the apparatus here comprises four major cooperating subassemblies namely, a reservoir subassembly 52 for containing the fluid to be dispensed to the patient, a flow control subassembly 54 for controlling the flow of fluid from the reservoir subassembly to the patient (FIG. 3), a fluid dispensing subassembly 56 (FIG. 2) for dispensing the fluid to the patient and a fill assembly 58 for controllably filling the reservoir with the fluid to be dispensed to the patient (FIG. 3). The details of each of these subassemblies, which are carried by a housing 60, will be discussed in greater detail in the paragraphs which follow.

Considering first the reservoir subassembly shown in FIG. 6, this subassembly includes a base assembly 62, a stored energy means, shown here as a distendable membrane 64, and a cover 66 for enclosing the stored energy source. The base assembly includes an ullage substrate 68 and a membrane capture housing 70 having a bottom opening 72 which receives the distendable membrane engaging element or protuberance 74 of base assembly 62 (see also FIG. 3). Distendable membrane 64 and ullage substrate 68 cooperate to define a fluid reservoir 75 for containing fluid to be dispensed to the patient. Reservoir 75 is provided with an inlet 75a for permitting fluid flow into said fluid reservoir and an outlet 75b for permitting fluid flow from said fluid reservoir.

Referring particularly to FIGS. 3 and 6, it can be seen that the ullage substrate 68 is provided with fill assembly receiving means shown here as a generally cylindrically-shaped receiving chamber 77 for receiving the connector portion of the fill assembly 58 (FIG. 3). Provided within chamber 77 is a pierceable septum 79 as well as the valve means of the invention, the nature and purpose of which will presently be discussed (FIG. 3).

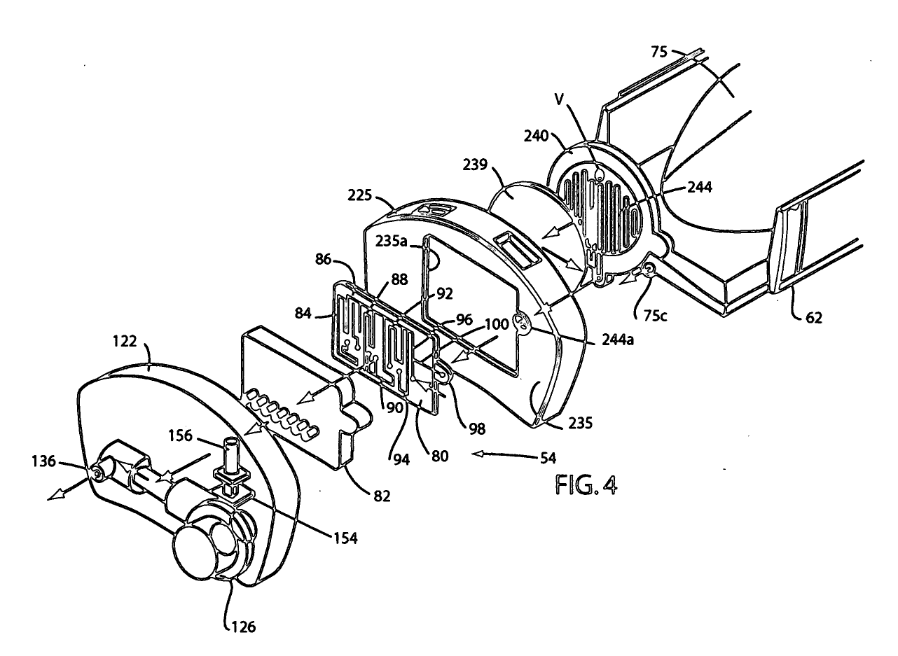

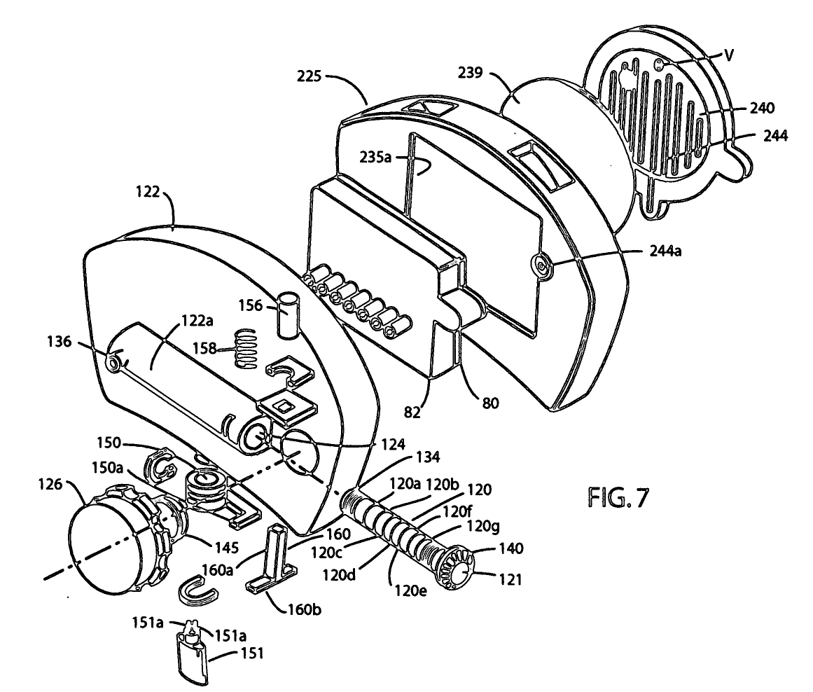

Considering next the important flow control means of the invention that comprises the novel flow rate control subassembly 54. This novel subassembly includes a novel flow rate control means that comprises a rate control base, plate or substrate 80 and an interconnected rate control cover 82 (FIG. 4). As best seen in FIGS. 4 and 20, rate control base, or plate 80 is uniquely provided with a plurality of fluidic micro-channels identified in the drawings as 84, 86, 88, 90, 92, 94 and 96. Each of the fluidic micro-channels is in communication with an inlet 98 via a filter means, or filter “F” and passageway 100 and each is provided with an outlet 102, 104, 106, 108, 110, 112 and 114 respectively. These outlets align with cover outlet ports 82a, 82b, 82c, 82d, 82e, 82f and 82g respectively (see FIG. 17) when the flow rate control assembly is assembled together in the manner illustrated in FIG. 7. Similarly, cover inlet port 82h aligns with rate control plate inlet 98 in the manner illustrated in FIG. 17. As will be presently described, each of the outlet ports formed in cover 82 can be placed in selective communication with the fluid dispensing means of the apparatus (FIG. 2) by controlled rotation of the selector member 120 of the rate control means of the invention the details of construction of which will presently be described.

It is to be understood that the micro-channels formed in rate control plate 80 may be of different sizes, lengths, widths, depths and configurations as shown by FIG. 19. Further, the flow control micro-channels may be rectangular in cross-section, or alternatively, they can be semicircular in cross-section, U-shaped in cross-section, or they may have any other cross-sectional configuration that may be appropriate to achieve the desired fluid flow characteristics. Additionally, as shown in FIG. 19A, the surface characteristics of the micro channels may be tailored to impart desired flow characteristics (for example, see surface coating “C”).

As indicated in FIG. 7, the flow rate control housing 122 of the flow control means is provided with an upraised portion 122a that defines an elongated, generally cylindrically-shaped chamber 124. Receivable within chamber 124 is the second portion of the flow rate control means of the invention, namely the selector means, which comprises previously identified selector member 120.

As best seen by referring to FIGS. 22 and 26, this important selector means of the invention also includes a cooperating control knob 126 which is used to controllably rotate selector member 120. As indicated in FIGS. 7, 14, 22 and 25, selector member 120 is provided with an axially-extending fluid flow passageway 128 and a plurality of radially-extending passageways 120a, 120b, 120c, 120d, 120e, 120f and 120g that communicate with passageway 128. In a manner presently to be described, rotation of selector member 120 within chamber 124 as a result of rotation of control knob 126 will permit a selected one of the plurality of radially-extending passageways formed in selector member 120 to be aligned with a selected one of the outlet ports of cover 82 and also with a selected one of the outlets of the fluidic micro-channels formed in rate control plate 80. As indicated in FIGS. 22 and 24, selector member 120 is provided with an outlet passageway 132, which communicates with axially-extending passageway 128 and also with a circumferentially-extending passageway 134. As indicated in FIG. 22, surrounding member 120 is sealing means, shown here as an elastomeric sleeve 12s which functions to seal member 120 relative to the housing. Circumferentially-extending passageway 134 communicates with an outlet port 136 formed on protuberance 122a (see FIG. 7), which, in turn, communicates with the fluid delivery line 138 of the fluid dispensing means (FIGS. 2, 7 and 15).

As indicated in FIGS. 7, 22 and 23, the proximal end 121 of selector member 120 is beveled and is provided with a plurality of circumferentially-spaced driven teeth 140. Teeth 140 mesh with a plurality of circumferentially-spaced driving teeth 142 formed on the inner beveled surface of a flange 144 of control knob 126 (see FIGS. 26 and 27). With this construction, when the shank portion 145 of control knob 126 is mated with flow control cover in the manner shown in FIG. 14, rotation of the control knob will impart rotation to the selector member 120. As previously mentioned, controlled rotation of selector member 120 will cause one of the radially-extending passageways formed within the selector member to be moved into fluid communication with a selected one of the outlets of the rate control channels formed in the rate control plate 80. As indicated in FIGS. 1 and 2, control knob 126 is provided with indicia “I” for indicating fluid flow rate toward the fluid delivery means of the apparatus.

Before further discussion of the operation of the selector means of the invention, the details of the construction of the rate control plate 80 and the various methods of making the rate control plate will now be considered. With respect to materials, the most appropriate materials for constructing the rate control plate are medical grade polymers. These types of polymers include thermoplastics, duroplastics, elastomers, polyurethanes, acrylics, silicones and epoxies. In other variations, the materials used for the flow control plate may be made of glass, silica or silicon. In further variations, the flow control component may be made of metals or inorganic oxides.

Using the foregoing materials, there are several ways that the flow control channels can be made. These include injection molding, injection-compression molding, hot embossing, casting and laser ablation. The techniques used to make these imbedded fluid channels are now commonplace in the field of microfluidics, which gave rise to the lab-on-a-chip, bio-MEMS and micro-total analysis systems (μ-TAS) industries. Additionally, depending on the size of the fluid channels required for a given flow rate, more conventional injection molding techniques can be used.

The first step in making the channels using an injection molding or embossing process is a lithographic step, which allows a precise pattern of channels to be printed on a “master” with lateral structure sizes down to 0.5 μm. Subsequently, electroforming is performed to produce the negative metal form or mold insert. Alternatively for larger channel systems, precision milling can be used to make the mold insert directly. Typical materials for the mold insert or embossing tool are nickel, nickel alloys, steel and brass. Once the mold insert or embossing tool is fabricated, the polymer of choice may be injection molded or embossed to yield the desired part with imprinted channels.

Alternatively, channels can be made by one of a variety of casting processes. In general, a liquid plastic resin, for example, a photopolymer can be applied to the surface of a metal master made by the techniques described in the preceding paragraph and then cured via thermal or ultraviolet (UV) means. After hardening, the material is then “released” from the mold to yield the desired part. Additionally, there are similar techniques available that utilize CAD data of the desired channel configuration and direct laser curing of a liquid monomer to yield a polymerized and solidified part with imbedded channels. This process is available by contract, from, by way of example, MicroTEC, GmbH of Duisburg, Germany.

In order to seal the flow control channels, a planar top plate may be used. In this instance, the channel system may be sealed with a top plate, which is here defined as any type of suitable cover that functions to seal the channel. The top plate may be sealably interconnected with the base plate which contains the flow channels by several means, including thermal bonding, sonic welding, laser welding, adhesive bonding and vacuum application.

Thermal bonding may be performed by using a channel base plate material and planar top cover that are made of similar polymeric materials. In this case the two substrates are placed in contact with one another, confined mechanically and heated to 2-5° C. above their glass transition temperature. Following a holding period sufficient enough for the polymer molecules of the two surfaces to interpenetrate with one another, the temperature is slowly reduced and a stress free bonded interface with imbedded micro-channels is yielded.

Additionally, the top plate may be bonded to the base plate through the use of one or more suitable bonding materials or adhesives. The bonding material or adhesive may be of the thermo-melting variety or of the liquid or light curable variety. For thermo-melting adhesives, the adhesive material is melted into the two apposed surfaces, thereby interpenetrating these surfaces and creating a sealed channel structure.

Further, liquid curable bonding materials or adhesives and light curable bonding materials or adhesives may be applied to one of the surfaces, for example the top plate. Subsequently, the other surface is brought into contact with the coated surface and the adhesive is cured by air exposure or via irradiation with a light source. Liquid curable bonding materials or adhesives may be elastomeric, for example, thermoplastic elastomers, natural or synthetic rubbers, polyurethanes, and silicones. Elastomeric bonding materials may or may not require pressure to seal the channel system. They may also provide closure and sealing to small irregularities in the apposed surfaces of the channel system.

A channel system may also be formed and sealed in cases where two surfaces are being joined and one of the surfaces has one or more apertures. In order to promote bonding between these two surfaces, a vacuum may be applied to the apertures. Bonding may then be accomplished by thermal methods or after previously having applied a bonding material or adhesive.

While the rate control plate can be constructed in various sizes, a rate control chip which is rectangular in shape and approximately 11 cm long and approximately 5 cm wide is suitable for the present application. Similarly, while the depth of the channels can vary depending upon the end use of the device, as a general rule the depth of the channels is on the order of approximately 1-1000 μm.

As previously mentioned, the cross section of the set of channels may vary in area over the members of the set of individual channels so as to achieve the specified flow rate of a particular channel. The cross section may also vary over the length of any particular channel so as to achieve the specified flow rate for the particular channel. Some examples of typical channel cross sections are square, rectangular, elliptical, circular, semi-circular and semi-elliptical. Channel cross sections may also be more complicated than those noted explicitly here.

A typical chip will be able to deliver fluid at multiple specified flow rates as, for example 0.25, 0.5, 1.0, 2.0 5.0 ml/hr. and greater for optimum performance, the flow rate should be constant and within 10% of the desired specified value at room temperature.

In operation, the flow through the flow control channels is controlled by taking advantage of the viscous drag imposed on the moving fluid by the walls of the channels. For a given imposed pressure and channel cross section, the longer the channel the smaller the flow rate. The pressure required to achieve the desired flow rates in the flow channels is preferably in the range of from 0.01 to 1 ATM. However, for some applications it may be desirable to exceed these limits.

The path that the micro-channels take in any given rate control plate may be straight, a single meander or two or more meanders. The turns of the meanders or serpentines may be of any angle from approximately 45° to approximately 220°. The runs of straight path between turns of the meanders may be of any length that the chip can accommodate, but these straight runs would typically be from 50 μm to 500 μm in length.

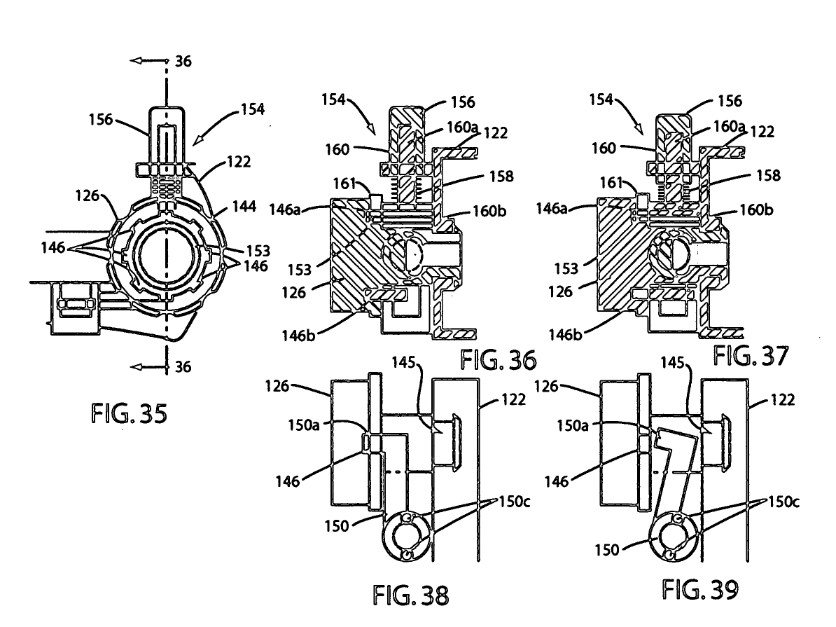

Another important feature of the invention resides in the provision of locking means for locking the selector knob in position after a particular fluid flow micro-channel has been selected through rotation of the selector knob. As indicated in FIGS. 26 and 35, flange portion 144 of control knob 126 is provided with a plurality of circumferentially-spaced-apart indexing cavities 146. Cavities 146 are adapted to receive the end of the outwardly extending finger portion 150a of a locking member 150 that is rotatably carried by flow control housing 122 for rotation by means of a physician's key 151 (see FIG. 7) between a first locked position shown in FIG. 38 and a second retracted position shown in FIG. 39. In the present form of the invention, the physician's key is provided with spaced-apart tangs 151a that are receivable within the spaced-apart bores 150c formed in locking member 150 (see FIGS. 7, 38 and 39). Once the end 150a of the locking member 150 is in the retracted position, novel release means are provided to permit knob 126 to be rotated to another position. In the present form of the invention this release means comprises a release assembly that is carried by flow control housing 122 in the manner best seen in FIGS. 7 and 36. Release assembly 154 (See FIGS. 4, 7, 36 and 37) here comprises a push member 156 that can be pushed downwardly in the manner shown in FIG. 37 against the urging of a coil spring 158. Disposed within push member 156 is a knob-locking member 160 which includes a shank portion 160a and an outwardly extending base portion 160b (FIG. 7). When push member 156 is in the upper position shown in FIG. 36, the outboard portion 161 of the base portion extends into an indexing cavity 146a formed in the control knob that is spaced 180° from the indexing cavity 146b that receives the extremity of arm 150a of locking member 150. When the push member is pushed into its downward position shown in FIG. 37, outboard portion 161 of the base portion moves from indexing cavity 146b into a circumferentially-extending groove 153 formed in control knob 126 (see FIGS. 35 and 37). When outboard portion 161 is moved into groove 153, knob 126 can be freely rotated to impart rotation to selector member 120 so as to permit another one of the plurality of radially-extending passageways formed in selector member 120 to be aligned with a selected one of the outlet ports of cover 82 and also with a selected one of the outlets of the fluidic micro-channels formed in rate control plate 80. Once knob 126 has been rotated into the desired position the downward pressure exerted, on member 156 is released causing spring 158 to once again move outboard portion 161 of the release means into a selected indexing cavity formed in knob 126 thereby once again locking the control knob against rotation. This done, using the physicians key, the caregiver can once again rotate member 150 into the locking position shown in FIG. 38. Through manipulation of the release means of the invention and the control knob in the manner previously described, it is apparent that the caregiver can select the desired rate of fluid flow from reservoir 75 to the patient via the administration set 163 of the fluid dispensing means (FIG. 2).

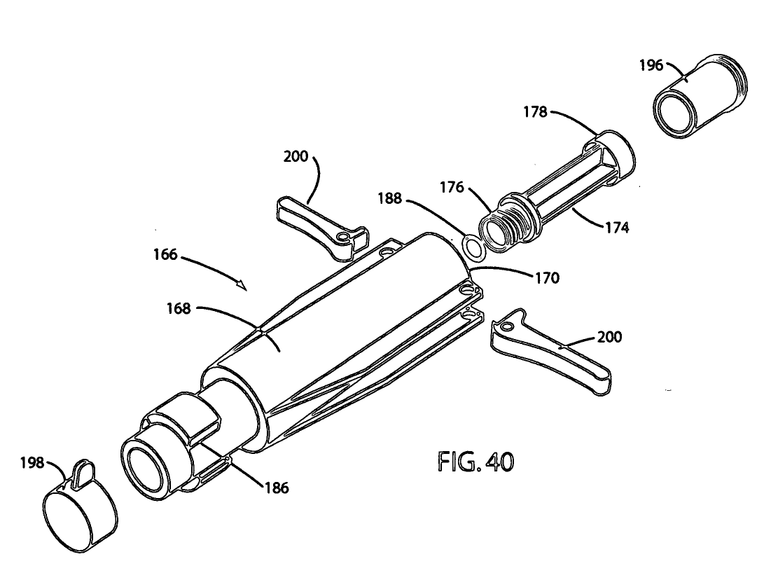

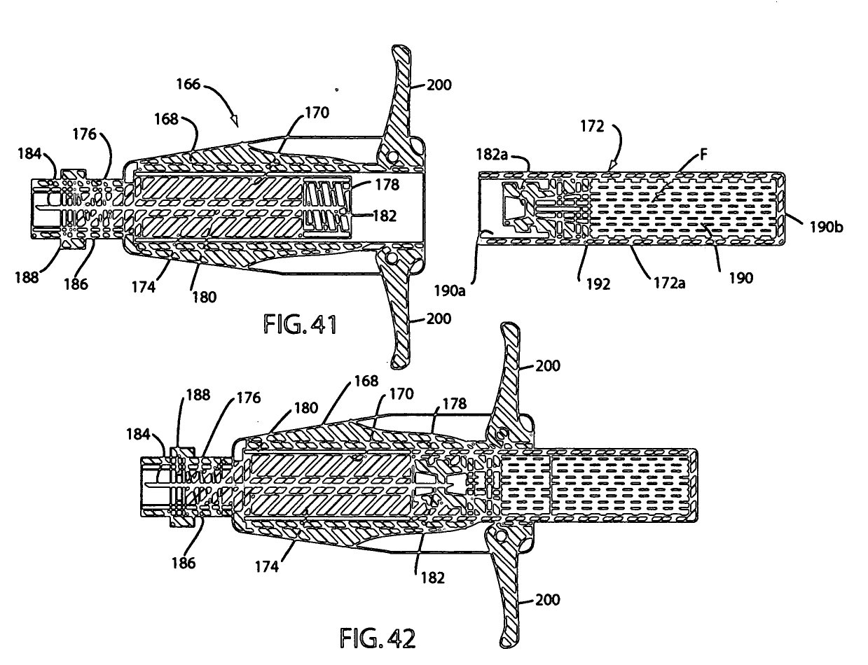

Consider next one form of the fill assembly 58 for controllably filling the reservoir with the fluid to be dispensed to the patient. As previously discussed and as shown in FIG. 3, ullage substrate 68 is provided with fill assembly receiving means shown here as cylindrically-shaped receiving chamber 77 that is adapted to receive in an aseptic condition the connector portion of the fill assembly 58. As illustrated in FIGS. 40 through 42, one form of the fill assembly of the invention comprises a syringe-type fill component 166 which includes a hollow housing 168 that is provided with a chamber 170 (FIG. 41) for telescopically receiving a medicament containing fill vial container 172 (FIG. 42), the construction of which will presently be described.

An elongated support 174, which is mounted within chamber 170 of component 168, includes threaded end portions 176 and 178 and a central flow passageway 180. Support 174 carries at one end a hollow needle 182 having a flow passageway which communicates, via passageway 180, with the flow passageway of a second needle or cannula 184 that is carried interiorly of the connector portion 186 of the fill means, or fill assembly 168. Portion 176 of support 174 is threadably interconnected within connector portion 186 and is sealed with respect thereto by means of an O-ring 188 (FIG. 41). Second cannula 184 is adapted to pierce the earlier identified septum 79 when the syringe assembly is operably interconnected with the base assembly 62 in the manner shown in FIG. 3. Septum 79 can be either a slit septum or a solid septum and is preferably constructed from an elastomeric material such as a silicone rubber. It is to be understood that a mechanical check valve can also serve as a septal interface. Such a valve is commercially available from C. R. Bard of Murray Hill, N.J.

Referring particularly to FIG. 41 of the drawings, the medicament containing fill vial 172 of this form of the invention, includes a body portion 172a, having a fluid chamber 190 for containing the injectable fluid medicament “F”. Chamber 190 is provided with a first open end 190a and second closed end 190b. First open end 190a is sealably closed by closure means here provided in the form of an externally threaded elastomeric plunger 192 which is telescopically movable within chamber 190 from a first location wherein the plunger is disposed proximate first open end 190a to the second, device-fill location, wherein the plunger is disposed proximate second closed end 190b.

After removal of a closure member 196 from the syringe assembly (FIG. 40), vial 172 can be inserted into chamber 170. As the fill vial is so introduced and the plunger 192 is threadably interconnected with threaded end 178 of support 174, the sharp end of the elongated needle 182 will pierce the central wall 182a of the elastomeric plunger in the manner shown in FIG. 42. Following removal of cover member 198, which covers connector portion 186 of the syringe assembly (FIG. 40), the assembly shown in FIG. 41 of the drawings can be mated with the fluid dispenser in the manner shown in FIG. 3. This done, the gripping fingers 200 can be moved from a retracted position to the extended position shown in FIGS. 41 and 42.

With the syringe fill assembly of the invention mated with the fluid dispenser in the manner shown in FIG. 3, the caregiver can grip the fingers 200 with his or her fingers and can exert an inward pressure on vial 172 causing the vial to move inwardly of chamber 170. A continuous movement of the vial into chamber 170 will cause the structural support 174 to move the elastomeric plunger inwardly of the vial chamber 190 in a direction toward the second or closed end 190b of the vial chamber. As the plunger is moved inwardly of the vial, the fluid “F” (FIG. 41) contained within the vial chamber will be expelled therefrom into the hollow elongated needle 180 (See FIG. 42). The fluid will then flow into hollow needle 184 which has pierced septum 79 and, as best seen in FIG. 3, will then flow past the valve means which is here shown as a conventional umbrella type check valve 204. The fluid will flow into inlet passageway 206 and then into reservoir 75.

A number of beneficial agents can be contained within vial container 172 and can be controllably dispensed to the patient including, by way of example, medicaments of various types, drugs, pharmaceuticals, hormones, antibodies, biologically active materials, elements, chemical compounds, or any other suitable material useful in diagnostic cure, medication, treatment or prevention of diseases or the maintenance of the good health of the patient.

As the fluid flows into reservoir 75, it will exert an inward pressure on the distendable membrane 64 distending it from the position shown in the solid lines in FIG. 3 to the position shown in the phantom lines in FIG. 3. Distendable membrane 64 can be in the form of a single pre-stressed or unstressed isotropic, elastomeric distendable membrane, or it can comprise a laminate assemblage made up of a plurality of initially generally planar distendable elements or films.

As indicated by FIG. 3, upstanding tongue 62a of base 62 extends completely about the perimeter of the base and is closely receivable within a groove 70a of capture housing 70. When the ullage substrate and the membrane capture housing are assembled in the manner shown in FIG. 3, the periphery of distendable membrane 64 will be securely clamped within groove 70a by tongue 62a. After the parts are thus assembled, capture housing 70 is bonded to base 62 by any suitable means such as adhesive or sonic bonding. This done, cover 66 is mated with capture housing 70 in the manner shown in FIG. 3 and bonded in place.

Upon opening the fluid delivery path, in a manner presently to be described, distendable membrane 64 will tend to return to its starting configuration thereby controllably urging fluid flow outwardly of the reservoir 75. The fluid will then flow, via the flow control means of the invention, into the dispensing means of the invention, which comprises the earlier identified conventional administration set 163 (FIG. 2). Administration set 163 is connected to housing 122 by a connector 211 in the manner shown in FIG. 2 of the drawings. The proximal end 213a of administration line 213 of the administration set is in communication with outlet 136 which is formed in housing 122 in the manner best seen in FIGS. 2 and 4. Disposed between the proximal end 213a and the distal end 213b of the administration line 213 is a conventional Y-site 215, a conventional gas vent and filter 217 and a conventional line clamp 219. Provided at the distal end 213b is a luer connector 221 of conventional construction (FIG. 2).

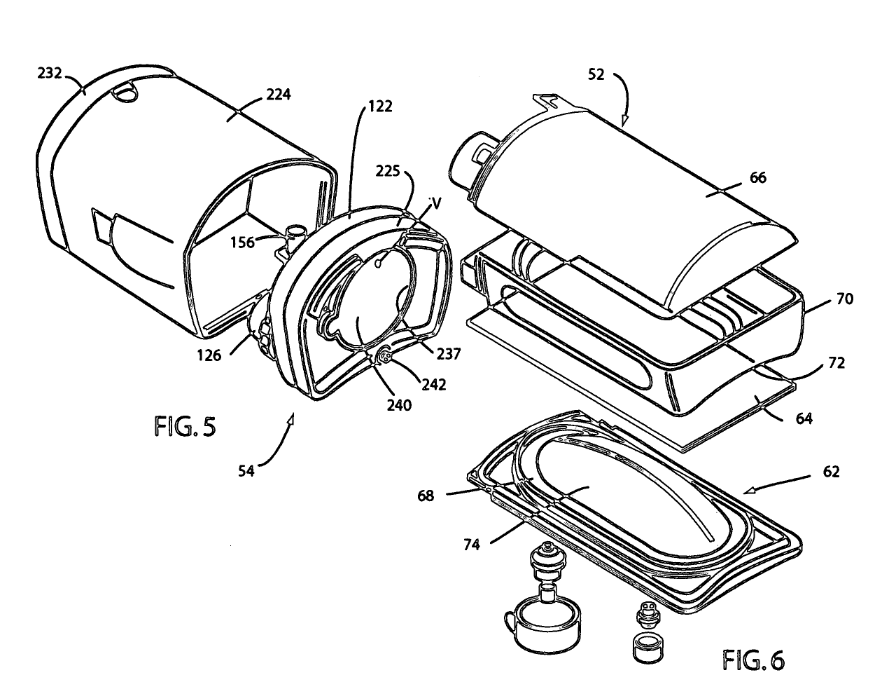

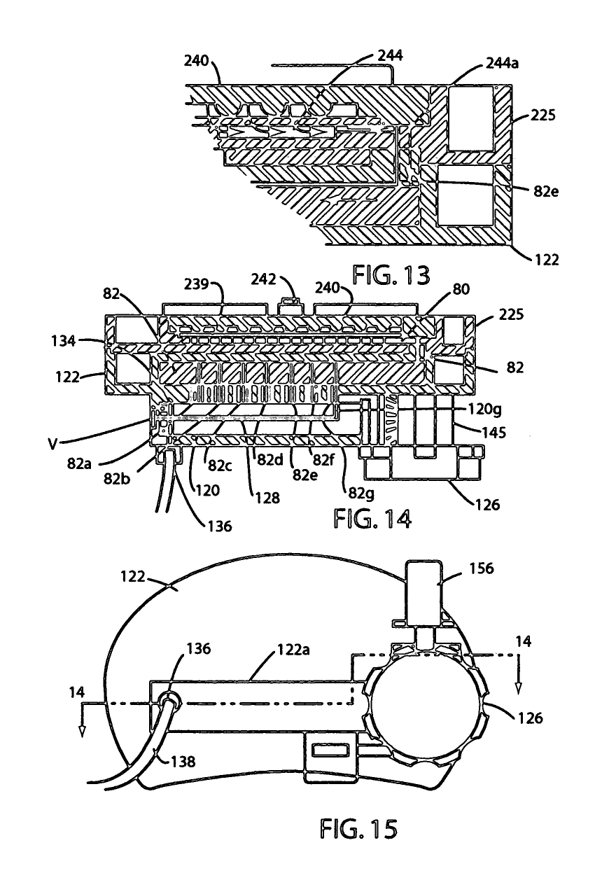

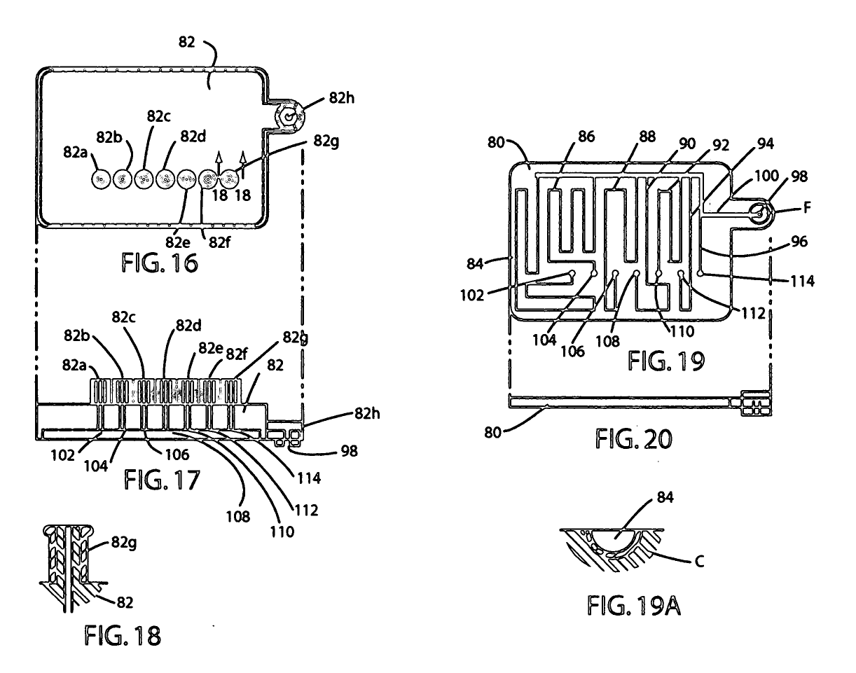

Turning now to a consideration of the important cover means of this latest form of the invention, this means here comprises a housing assembly 224 which is interconnected with the reservoir subassembly 52 and functions to close the forward or delivery end of the device (see FIGS. 1, 2 and 3). As best seen in FIGS. 3 and 4, housing assembly 224 includes the previously identified flow rate control housing 122 which defines a first compartment 226 that houses the flow rate control plate 80 and cover 82 and a second compartment 228 that houses the selection means, including the control knob and locking means of the invention. A third compartment 230 is defined by a cover component 232 that is pivotally movable from the closed position shown in FIG. 1 to the open position shown in FIG. 2. Compartment 230 functions to house the dispensing means, or administration set 163 of the invention, when the administration set is not in use. As best seen in FIG. 5, rear face 235 of housing assembly 225 has a centrally disposed, socket-like recess 237 that closely receives a filter means shown here as a conventional particulate filter 239 and an inlet, or dispersion element, 240 when structure 225 is mated with reservoir subassembly 52 in the manner shown in FIG. 3 of the drawings. Inlet element 240, which functions as a fluid dispersion element, includes an inlet 242, which communicates with the outlet 75b of fluid reservoir 75 via a flow passageway 75c (FIG. 3). Inlet 242 also communicates with a circuitous fluid passageway 244, which has an outlet 244a (see FIGS. 4 and 13) that, in turn, communicates with inlet 82h to cover 82 of the flow rate control assembly (see FIG. 16). Face 235 also has a rectangular opening 235a which receives the rate control plate 84 of the flow control subassembly 54 (see FIG. 4).

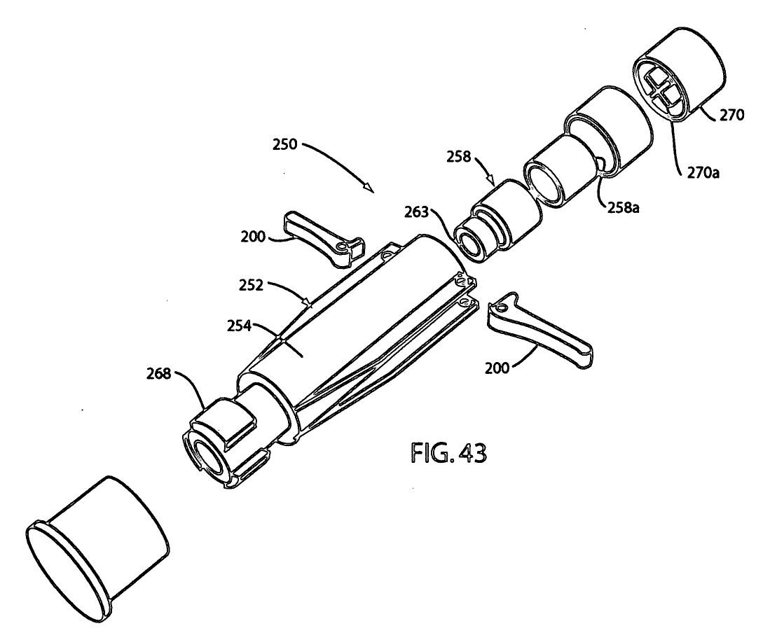

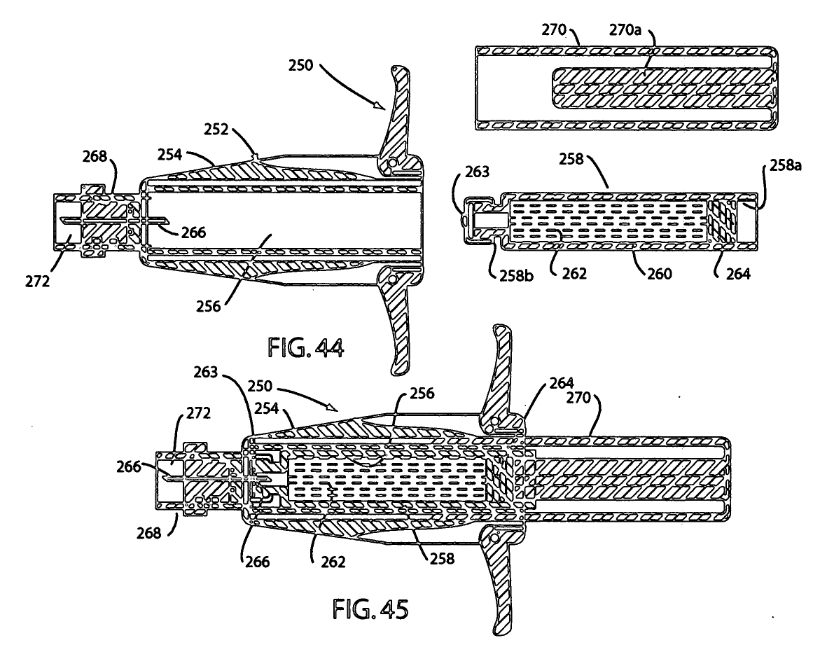

Referring next to FIGS. 43, 44 and 45, an alternate form of the fill means of the invention is there shown and generally designated by the numeral 250. This alternate form of fill means is similar in many respects to that shown in FIGS. 40, 41 and 42 and like numerals are used to identify like components. As shown in FIG. 44 this alternate form of fill means comprises a syringe-type fill component 252 which includes a hollow housing 254 that is provided with a chamber 256 (FIG. 44) for telescopically receiving a medicament containing cartridge fill vial container 258 the construction of which is illustrated in FIG. 44.

As shown in FIG. 44, cartridge fill vial 258 comprises a hollow glass or plastic body portion 260 that defines a fluid chamber 262. Fill vial 258 has an open first end 258a and a second end 258b that is closed by a pierceable, elastomeric septum 263. An elastomeric plunger 264 is reciprocally movable within fluid chamber 262. As shown in FIG. 44, a hollow needle 266 is mounted within the connector portion 268 of the hollow housing 254. Hollow needle 266 is adapted to pierce septum 263 when the fill vial is inserted into a chamber 256 and pushed into the position shown in FIG. 45 by the pusher means, or pusher assembly 270. With this construction, as the fluid contained within the fluid chamber 262 is urged outwardly thereof by pusher 270a (See FIG. 43) of the pusher assembly 270 fluid will controllably flow into hollow needle 266.

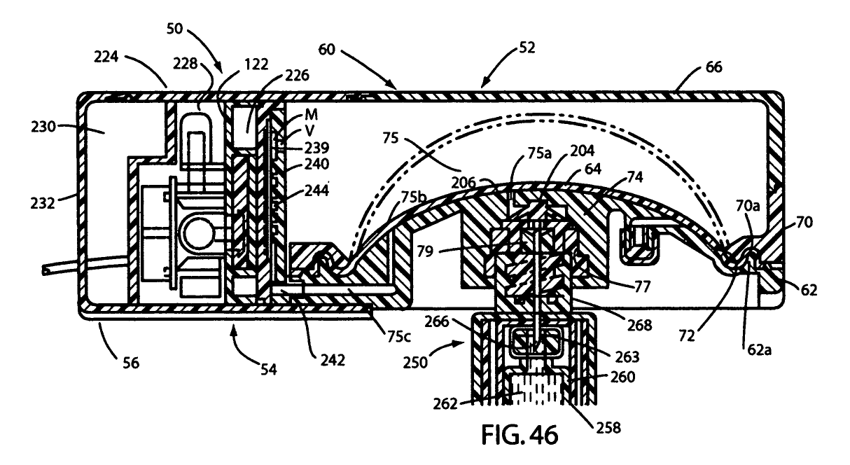

Turning to FIG. 46, it can be seen that when the fill means 250 is mated with the fluid dispenser, needle 266 pierces septum 79 which permits the fluid contained within the fluid chamber 262 to flow into cavity 79, past umbrella type check valve 204 and into reservoir 75 via inlet 75a.

A number of beneficial agents can be contained within vial 258 and can be controllably dispensed to the patient including, by way of example, medicaments of various types, drugs, pharmaceuticals, hormones, antibodies, biologically active materials, elements, chemical compounds, or any other suitable material useful in diagnostic cure, medication, treatment or prevention of diseases or the maintenance of the good health of the patient.

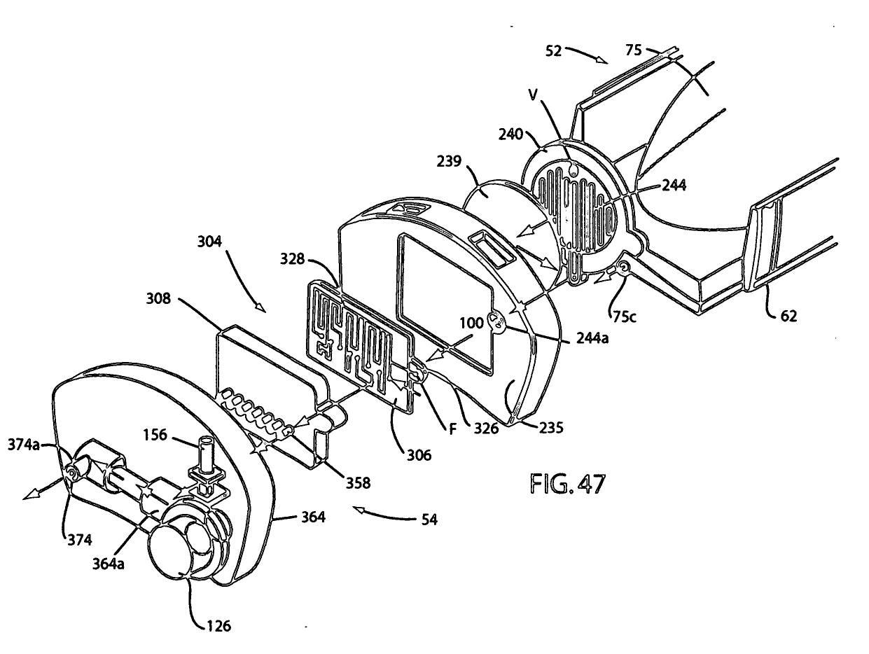

In operation of the apparatus of the invention to deliver medicinal fluids to the patient at a controlled rate, following the opening of the fluid delivery path, distendable membrane 64 will tend to return to its starting configuration thereby controllably urging fluid flow outwardly of the reservoir 75. The fluid will flow from the reservoir, through reservoir outlet port 75b, into inlet 242 of dispersion element 240, through circuitous fluid passageway 244, through particulate filter 239, through outlet 244a and into inlet 326 of the control subassembly 54 (see FIG. 47). From inlet 326 the fluid will flow via filter means, here provided as a filter “F” (see FIGS. 49 and 58) into each of the micro-channels of the rate control plate 80.

When the selector knob 126 is in the priming position the fluid will flow from micro-channel 96 into radial passageway 120g of selector member 120, into axial passageway 128, then into an annular passageway 134, which is in communication therewith and toward outlet port 136 formed on protuberance 122a (see FIG. 7). During this process any gases contained within the fluid passageways will be vented to atmosphere via the vent means “V” (FIG. 14).

Delivery of fluid to the patient at different selected rates can be accomplished in a similar manner through rotation of knob 126 and selector member 302 to align other radial passageways of the selector member with selected outlets of the micro-channels of the rate control plate 80.

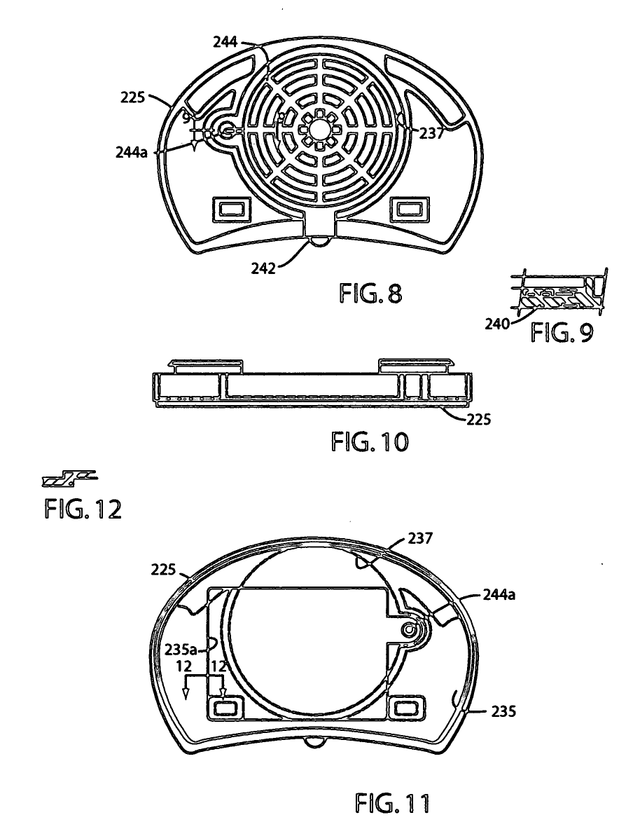

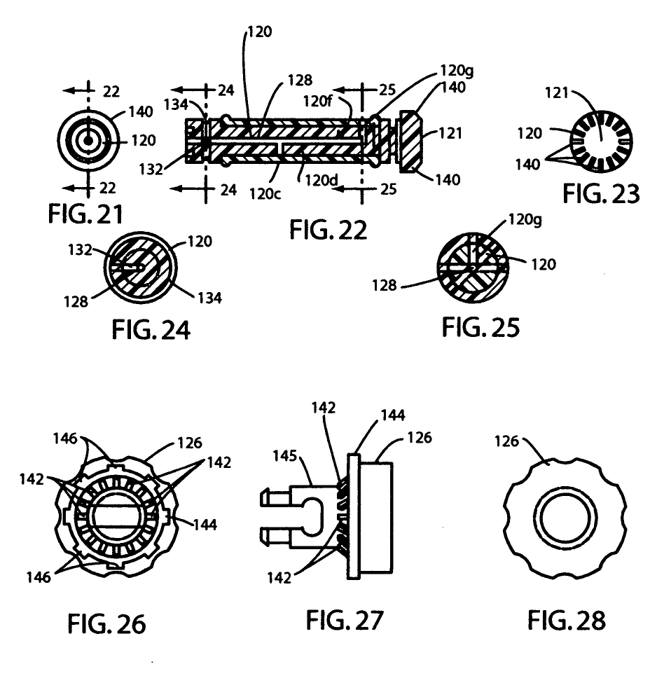

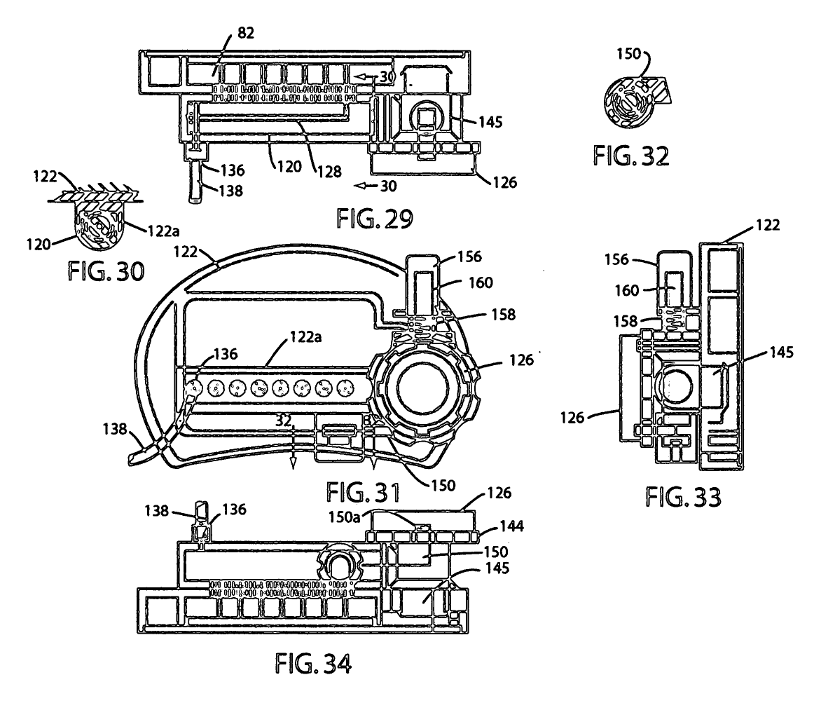

Referring next to FIGS. 47 through 58, a portion of an alternate form of the apparatus of the invention is there shown. This alternate form of the apparatus is similar in many respects to that shown in FIGS. 1 through 46 and like numerals are used in FIGS. 47 through 59 to identify like components. A primary difference between this latest form of the invention and that earlier described herein resides in the provision of flow rate control means which uniquely includes priming means for priming the various fluid passageways of the device prior to delivery of fluid to the administration set.

As best seen in FIG. 47, the apparatus of this latest form of the invention comprises four major cooperating subassemblies namely, a reservoir subassembly 52 for containing the fluid to be dispensed to the patient, a flow control means for controlling the flow of fluid from the reservoir subassembly to the patient, a fluid dispensing subassembly 56 for dispensing the fluid to the patient and a fill assembly, similar to fill assembly 250 (FIG. 46), for controllably filling the reservoir with the fluid to be dispensed to the patient.

The reservoir subassembly 52, the fluid dispensing subassembly 56 and the fill assembly 250 are substantially identical in construction and operation to those previously described herein and the details of their construction will not be further described. However, as previously discussed, the important flow control means of the invention for controlling the rate of fluid flow toward the fluid dispensing subassembly 56 is somewhat different from that previously described in that it uniquely comprises a priming means for purging and priming the various passageways of the device prior to delivery of fluid from the fluid reservoir to the fluid dispensing subassembly 56. More particularly, this important priming means first purges to atmosphere any gases contained within the fluid passageways of the device and then controllably fills the fluid passageways with fluids drawn from the device reservoir. This feature of the apparatus ensures that only the desired fluid is delivered at the outlet passageway of the device during normal operation and that the device is in a state in which it will deliver fluid to the outlet passageway in as short a time as possible.

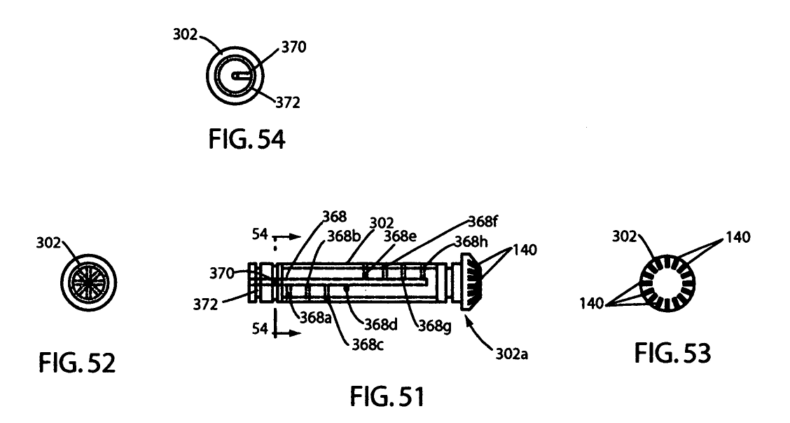

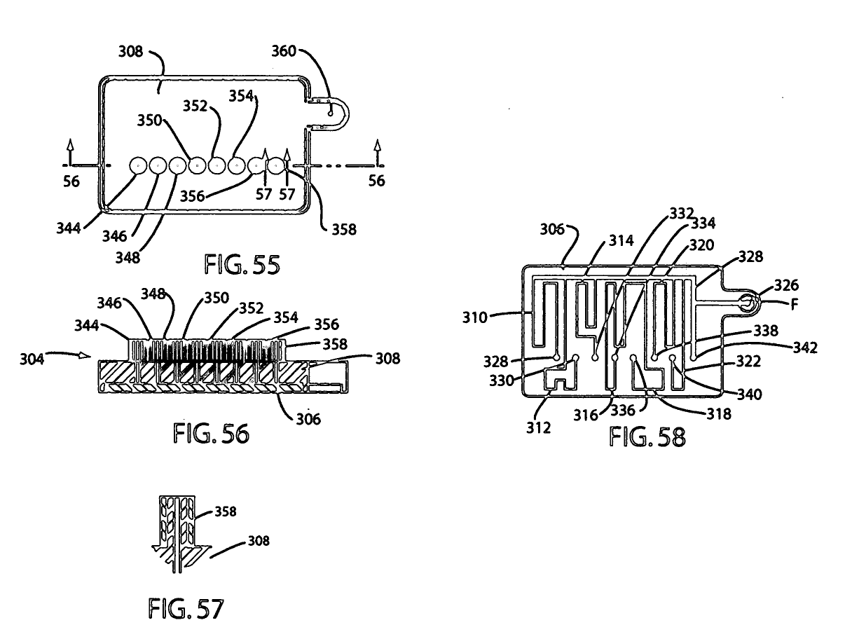

The novel flow control means of this latest form of the invention comprises a selector means, which includes a selector member 302 having a plurality of fluid passageways formed therein (FIG. 51) and a flow rate control assembly 304 (FIG. 56) for controlling the rate of fluid flow toward the fluid dispensing subassembly 56. Flow rate control assembly 304 includes a rate control plate, or member 306, and an interconnected rate control cover 308 (FIGS. 55 and 56). As best seen in FIGS. 47 and 58, rate control plate 306 is uniquely provided with a plurality of fluidic micro-channels identified in the drawings as 310, 312, 314, 316, 318, 320 and 322. Each of the fluidic micro-channels is in communication with an inlet 326 via a priming passageway 328, which comprises a part of the priming means of the invention, and each is provided with an outlet 328, 330, 332, 334, 336, 338, 340 and 342 respectively. These outlets align with cover outlet ports 344, 346, 348, 350, 352, 354, 356 and 358 respectively (see FIGS. 55, 56 and 58) when the flow rate control assembly is assembled together in the manner illustrated in FIG. 56. Similarly, cover inlet port 360 aligns with rate control plate inlet 326 in the manner depicted in the drawings. As in the earlier described embodiment of the invention, each of the outlet ports formed in cover 308 can be placed in selective communication with the fluid dispensing means of the apparatus by controlled rotation of the selector member 302 of the rate control means of the invention the details of construction of which will presently be described.

It is to be understood that, as before, the micro-channels formed in rate control plate 306 may be of different sizes, cross-sectional areas, lengths and configurations as shown by FIG. 58. Further, the flow control micro-channels may be rectangular in cross-section, or alternatively, they can be semicircular in cross-section, U-shaped in cross-section, or they may have any other cross-sectional configuration that may be appropriate to achieve the desired fluid flow characteristics.

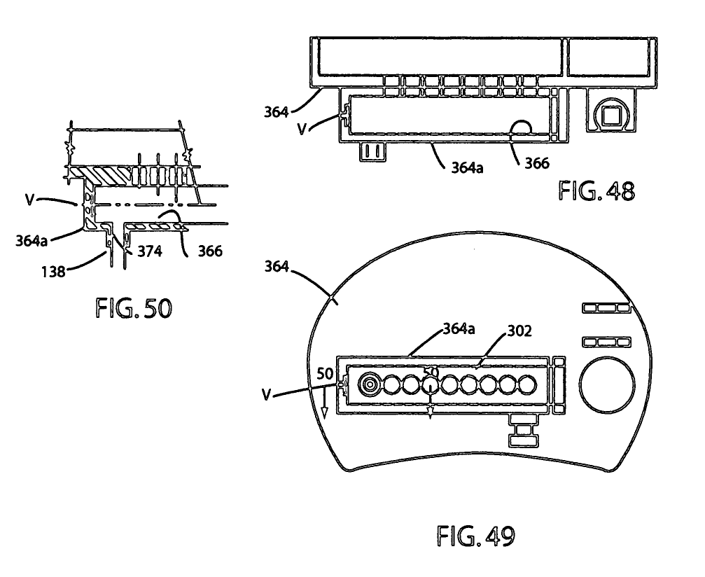

As indicated in FIG. 48, the flow rate control housing 364 of the flow control means is provided with an upraised portion 364a that defines an elongated, generally cylindrically-shaped chamber 366. Receivable within chamber 366 is the second portion of the flow control means of the invention, namely the selector means, which comprises the previously identified selector member 302. As before, sealing means in the form of an elastomeric sleeve 302s circumscribes member 302 and functions to seal member 302 relative to chamber 366.

Referring to FIG. 47, it can be seen that the important selector means of this latest embodiment of the invention also includes a cooperating control knob 126 which is used to controllably rotate selector member 302. As indicated in FIGS. 51, 52, 53 and 54, selector member 302 is provided with an axially-extending fluid flow passageway 368 and a plurality of radially-extending passageways 368a, 368b, 368c, 368d, 368e, 368f, 368g and 368h that communicate with passageway 368. In a manner presently to be described, rotation of selector member 302 within chamber 366 as a result of rotation of control knob 126 will permit a selected one of the plurality of radially-extending passageways formed in selector member 302 to be aligned with a selected one of the outlet ports of cover 308 and also with a selected one of the outlets of the fluidic micro-channels formed in rate control plate 306. As indicated in FIGS. 51 and 54, selector member 302 is provided with an outlet passageway 370, which communicates with axially-extending passageway 368 and also with a circumferentially-extending passageway 372. Circumferentially-extending passageway 372 communicates with an outlet port 374 formed on protuberance 364a (see FIG. 50), which, in turn, communicates with the fluid delivery line 138 of the fluid dispensing means (FIGS. 2, 7 and 15).

As shown in FIG. 51, the proximal end 302a of selector member 302 is beveled and is provided with a plurality of circumferentially-spaced driven teeth 140. Teeth 140 mesh with a plurality of circumferentially-spaced driving teeth 142 formed on the inner beveled surface of a flange 144 of control knob 126 (see also FIGS. 26 and 27). With this construction, when the shank portion 145 of control knob 302 is mated with flow control cover in the manner shown in FIG. 47, rotation of the control knob will impart rotation to the selector member 302. As previously mentioned, controlled rotation of selector member 302 will cause one of the radially-extending passageways formed within the selector member to be moved into fluid communication with a selected one of the outlets of the rate control channels formed in the rate control plate 306.

Another important feature of the invention resides in the provision of locking means for locking the selector knob in position after a particular fluid flow micro-channel has been selected through rotation of the selector knob. The locking means of this latest form of the invention is identical in construction and operation to that previously described.

Similarly, the fill assembly of this latest form of the invention for controllably filling the reservoir with the fluid to be dispensed to the patient is identical in construction and operation to that described in connection with the embodiment of the invention shown in FIGS. 1 through 46.

Upon opening the fluid delivery path of this latest form of the invention, distendable membrane 64 (FIG. 3) will tend to return to its starting configuration thereby controllably urging fluid flow outwardly of the reservoir 75 (FIG. 3). The fluid will then flow through reservoir outlet port 75b, into the inlet of dispersion element 240, through circuitous fluid passageway 244, through particulate filter 239, through outlet 244a and into inlet 326 of the flow rate control assembly (see FIG. 47). From inlet 326 the fluid will flow into priming channel 328 via the filter “F” as well as into each of the micro-channels of the rate control plate 306.

When the selector knob 126 is in the priming position shown in FIG. 47, the fluid will flow from a priming channel 328 into radial passageway 368h of selector member 302, into axial passageway 368 and toward outlet 374 thus priming these passageways with fluid and to purge any gases contained therein to atmosphere via the vent means “V” (FIG. 50).

By way of example, when the selector knob 126 is rotated to a position wherein radial passageway 368g of selector member 302 is aligned with the outlet 340 of micro-channel 322 of the rate control plate 306, fluid will flow from micro-channel 322 into passageway 368, then into annular passageway 372 which is in communication therewith and then into outlet 374 at a precisely controlled rate (FIGS. 47, 51 and 58). Delivery of fluid to the patient at different selected rates can be accomplished in a similar manner through rotation of knob 126 and selector member 302 to align other radial passageways of the selector member with selected outlets of the micro-channels of the rate control plate 306.

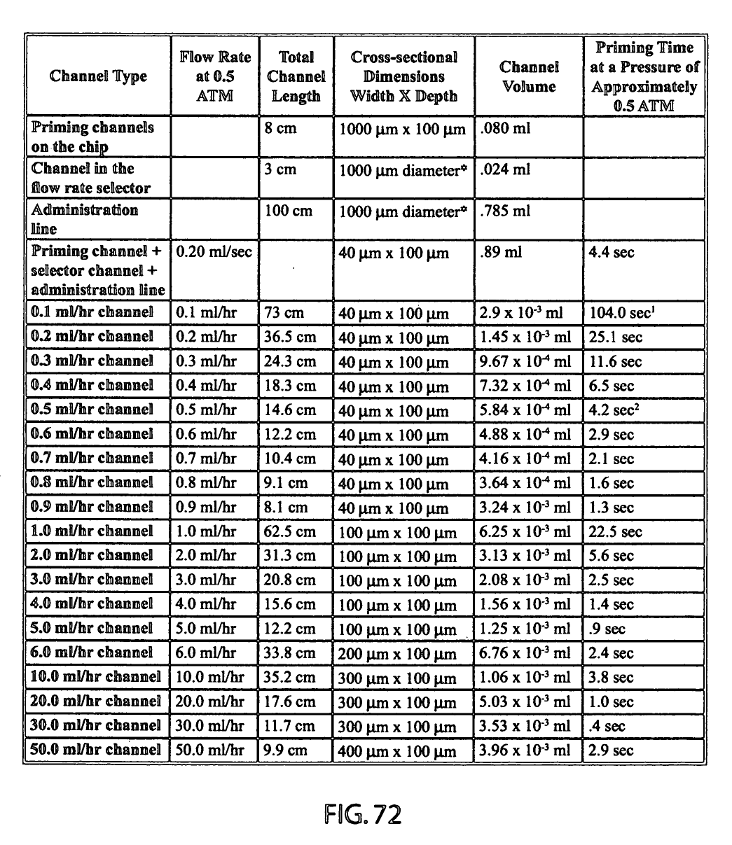

It is important to note that priming of the various fluid passageways of the device ensures that only the desired fluid is delivered at the output of the device during normal operation and that the device is in a state in which it will deliver fluid at the exit of the administration line in a reasonably short a time. The value of the priming means of this latest form of the invention is evident from a study of FIG. 72 of the drawings which comprises a table of the fluidic properties of one form of the flow rate control member, or chip 306, the flow rate selector means and the administration line of the device of this latest form of the invention. For purposes of illustration in FIG. 72, the flow rates are shown to be between 0.1 and 50 ml/hr and the rate defining channels are assumed to be from 4000 μm² to 40,000 μm². Similarly, the priming channel is assumed to be 1000 μm×100 μm wide×deep, the channel in the rate control selector means is assumed to be 1 mm in diameter and 3 cm long and the administration line is assumed to be 1 meter long and 40 thousandths of an inch (approx. 1 mm) in diameter. The priming channels on the chip, the channel in the flow rate selector means and the administration line are treated as one item for the purpose of priming time and flow rate.

If the fluidic system is not compatible with the fluid being transported, either in terms of its biocompatibility or hyrdophilicity characteristics, a surface modification process will be needed. While not wanting to be held to a particular approach, the surface modification methodology may take one of several forms. One process that is extremely clean, fast and effective is plasma processing. In particular this technique allows for any of the following 1) plasma activation, 2) plasma induced grafting and 3) plasma polymerization of molecular entities on the surface of the bellows. For cases where an inert hydrophobic interface is desired, plasmas using hydrophilic molecules may be employed. That is, the channels' surface may be cleaned with an inert gas plasma, and subsequently, an appropriate plasma may be used to graft these molecule to the surface. Alternatively, if a hydrophobic surface is desired (e.g. for solutions that are highly corrosive or in oil-based solvents) an initial plasma cleaning may be done, followed by a plasma polymerization using hydrophobic monomers.

From a study of FIG. 72 it can be seen that if one of the flow rate defining fluidic micro-channels were used to prime the administration line, then there would be an unreasonably long time between the time that the device is initially “turned on” and the time that fluid is delivered from the administration line. This is because the volume of the administration line is 0.785 ml. For example, suppose the flow rate is 0.5 ml/hr then it would be 94 minutes (i.e., 0.785 ml/0.5 ml/hr=1.57 hours) before fluid emerges from the administration line and the device is ready to use. This length of time to wait before the device is ready to use is undesirable in most applications of the device. It is evident that a priming means envisioned by this latest form of the device of the invention is an advantageous feature which enables the device be ready to administer fluid in a matter of a minute or less.

Turning next to FIGS. 60 through 71, an alternate form of flow rate control assembly is there illustrated and generally designated by the numeral 440. Flow rate control assembly 440 is usable with the apparatus shown in FIGS. 4 and 7 of the drawings and is adapted to be disposed within chamber 226 of the device housing. This alternate form of the flow rate control assembly is also adapted to cooperate with the selector means of the apparatus of FIG. 4 in a manner previously described to select the desired rate of fluid flow from the fluid source toward the fluid delivery line.

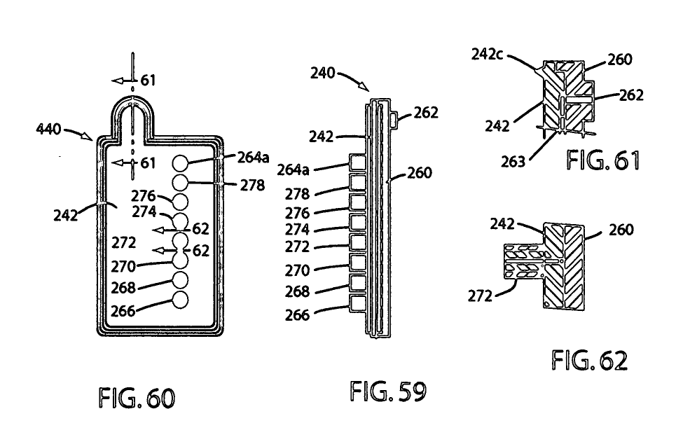

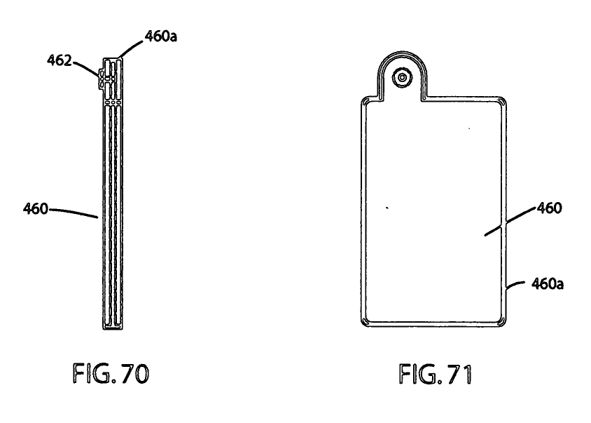

The primary difference between this latest flow rate control assembly and that previously described is that the fluidic micro flow channels which control the rate of fluid flow are formed in the lower surface 440a of the rate control cover 242 of the assembly (see FIG. 65). More particularly, lower surface 442a of cover 442 is provided with a plurality of micro channels identified as 444, 446, 448, 450, 452, 454, and 456. When the rate control base 460 of a rate control assembly is sealably interconnected with cover 442 in the manner shown in FIG. 59 the plurality of micro channels will be sealed to form a plurality of fluidic micro channels. In this regard, it is to be noted that a circumferentially-extending channel 442b is formed in cover 442 (FIG. 62). It is also to be observed that cover 442 is provided with a circumferentially extending, sonic energy director 442c (FIG. 66), which enables the cover member to be sonically bonded to the apparatus housing 122 when the alternate form of rate control assembly is positioned within chamber 226. Sealably receivable within channel 442b is an upstanding, circumferentially extending step 460a formed on base member 460 (FIGS. 70 and 71).

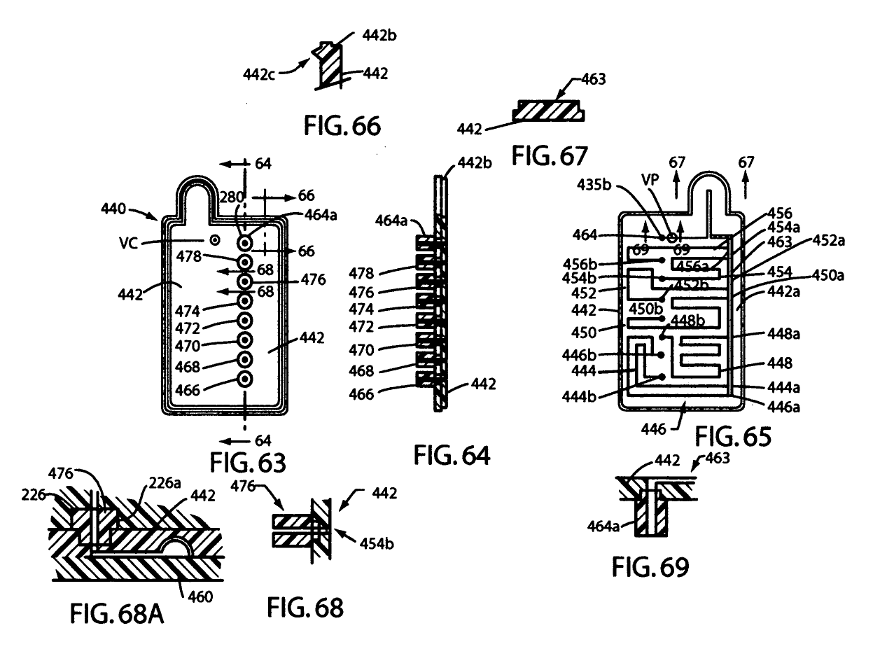

Each of the fluidic micro channels is in communication with the rate control inlet 462 via the priming means of the invention for purging and priming the various fluid delivery passageways of the flow control means. This priming means here comprises a prime channel 463 which functions to purge gases from delivery line 213 and to prime the various fluidic elements of the device before the fluid is delivered to the fluid delivery line 213. It is to be noted that the fluidic micro channels are provided with inlets 444a, 446a, 448a, 450a, 452a, 454a, and 456a respectfully (FIG. 65). These inlets are in communication with prime channel 463 so that as the prime channel is filled, each of the fluidic micro channels will also fill. Prime channel 463 is also in communication with a prime channel outlet port 464, which, in turn, communicates with cover outlet port 464a (FIG. 65) formed in cover member 442. Cover member outlet port 264a aligns with an inlet to the flow rate control assembly, the details of construction of which were described in connection with a description of the previously embodiment of the invention. As the various fluid flow passageways of the device fill with fluid during the priming step, gases contained within the passageways will be vented to atmosphere via a vent “V” formed in member 464a (FIG. 50). Additionally, venting can be provided by vent means formed on the fluidic chip or plate 460 in the form of a vent VP (FIG. 65) and on the cover 442 in the form of a vent VC (FIG. 63).

The fluidic micro channels are also provided with outlets 444b, 446b, 448b, 450b, 452b, 454b, 456b and 458b respectfully (FIG. 65). These outlets align with cover outlet ports 466, 468, 470, 472, 474, 476, and 478 respectively (FIG. 63). Each of the cover outlet ports comprises a compressible elastomeric sleeve which sealably engages the wall 226a of chamber 226 which receives the rate control assemblage 440 when the components are assembled in the manner shown in FIG. 68A. As the components are assembled, the sleeves are compressed to provide a fluid seal, or sealing means, that prevents fluid leakage about the ports.

As previously discussed in connection with the earlier described embodiment of the invention, each of the outlet ports formed in the rate control cover can be placed in selective communication with the fluid delivery line 213 by manipulation of the rate control means of the invention. In this way, the rate of fluid flow toward the fluid delivery line can be can be precisely controlled by the caregiver.

As earlier described herein, the fluidic micro channels formed in cover 142 of this latest form of the invention may be of different sizes, lengths and configurations as shown in FIG. 65. Further, the flow control fluidic micro channels may be rectangular in cross-section, or alternatively, can be semicircular in cross-section, U-shaped in cross-section, or they may have any other cross-sectional and surface configuration that may be appropriate to achieve the fluid flow characteristics that are desired in the particular end use application.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

We claim:

1. A device for use in infusing medicinal fluid into a patient at a controlled rate comprising:
(a) a housing including a base;
(b) stored energy means for forming, in conjunction with said base, a fluid reservoir having an inlet and an outlet, said stored energy means comprising at least one distendable member superimposed over said base, said member being distendable as a result of pressure imparted by the fluids to be infused, to establish internal stresses, said stresses tending to move said member toward a less distended configuration;
(c) fluid delivery means in communication with said outlet of said fluid reservoir for delivering fluid from the device;
(d) flow rate control means disposed between said outlet of said fluid reservoir and said fluid delivery means for controlling the rate of fluid flow toward said fluid delivery means, said flow rate control means comprising:
(i) a selector member rotatably carried by said housing, said selector member having a plurality of fluid passageways formed therein; and
(ii) a flow rate control assembly disposed between said outlet of said fluid reservoir and said selector member, said flow control assembly comprising a rate control base and a rate control cover connected to said base, one of said rate control base and said rate control cover having a plurality of elongated fluidic flow control channels in communication with said plurality of fluid passageways formed in said selector member; and
(e) fill means connected to said housing for filling said reservoir.

2. The apparatus as defined in claim 1 in which said fluid delivery means comprises an administration set and in which said housing includes a storage compartment for storing said administration set.

3. The apparatus as defined in claim 1 in which said flow rate control means further comprise priming means for priming said plurality of fluid passageways formed in said one of said flow control base and said flow control cover and in said selector member.

4. The apparatus as defined in claim 1, further including selector means carried by said housing for controllably rotating said selector member, said selector means comprising a control knob operably interconnected with said selector member.

5. The apparatus as defined in claim 4, further including locking means carried by said housing for preventing rotation of said control knob.

6. The apparatus as defined in claim 4 in which said housing further includes a connector portion and in which said fill means comprises a fill assembly interconnectable with said connector portion of said housing.

7. The apparatus as defined in claim 6 in which said fill assembly comprises a syringe assembly including:
(a) a hollow housing having a chamber; and
(b) a fill vial telescopically receivable with said chamber of said hollow housing, said fill vial having a fluid reservoir and a plunger disposed within said fluid reservoir for movement between first and second positions.

8. The apparatus as defined in claim 7 in which said connector portion includes valve means for controlling fluid flow toward said reservoir.

9. The apparatus as defined in claim 7 in which said connector portion includes a pierceable septum.

10. A device for use in infusing medicinal fluid into a patient at a controlled rate comprising:
(a) a housing including a base provided with a connector portion;
(b) stored energy means for forming, in conjunction with said base a fluid reservoir having an inlet and an outlet, said stored energy means comprising a distendable membrane superimposed over said base, said membrane being distendable as a result of pressure imparted by the fluids to be infused, to establish internal stresses, said stresses tending to move said membrane toward a less distended configuration;
(c) fluid delivery means in communication with said outlet of said fluid reservoir for delivering fluid from the device;
(d) flow rate control means disposed between said outlet of said fluid reservoir and said fluid delivery means for controlling the rate of fluid flow toward said fluid delivery means, said flow rate control means comprising:
(i) a selector member rotatably carried by said housing, said selector member having a plurality of fluid passageways formed therein;
(ii) a flow rate control base disposed between said outlet of said fluid reservoir and said selector member, said flow rate control base having a plurality of elongated fluidic flow control channels in communication with said plurality of fluid passageways formed in said selector member;
(iii) selector means carried by said housing for controllably rotating said selector member, said selector means comprising a control knob operably interconnected with said selector member; and
(iv) priming means for priming said plurality of fluid passageways formed in said flow control member and in said selector member, and
(e) fill means connected to said housing for filling said reservoir, said fill means comprising a fill assembly interconnectable with said connector portion of said housing.

11. The apparatus as defined in claim 10 in which said fluid delivery means comprises an administration set and in which said housing includes a storage compartment for storing said administration set.

12. The apparatus as defined in claim 10, in which said plurality of elongated fluidic flow control channels of said flow rate control member have a depth of between about 1 μm and about 1000 μm.

13. The apparatus as defined in claim 10 in which said fill assembly comprises a syringe assembly including:
(a) a hollow housing having a chamber; and
(b) a fill vial telescopically receivable with said chamber of said hollow housing, said fill vial having a fluid reservoir and a plunger disposed within said fluid reservoir for movement between first and second positions.

14. The apparatus as defined in claim 10 in which said connector portion of said base includes valve means for controlling fluid flow toward said reservoir.

15. The apparatus as defined in claim 10 in which said connector portion includes a pierceable septum.

16. The apparatus as defined in claim 10 in which said rate control means includes sealing means for substantially sealing said selector member relative to said housing.

17. The apparatus as defined in claim 10 in which said fluidic flow control channels have surfaces and in which said surfaces are tailored to impart certain surface characteristics.

18. The apparatus as defined in claim 10 in which said flow rate control means further comprises a cover connected to said rate control base, said cover having outlet ports comprising compressible elastomeric sleeves.

19. The apparatus as defined in claim 10 further including filter means for filtering the fluid flowing from said fluid reservoir toward said fluidic flow control channels.

20. The apparatus as defined in claim 10 in which said rate control means further comprises vent means for venting to atmosphere gases contained with said fluidic flow control channels.

21. The apparatus as defined in claim 10, further including locking means carried by said housing for preventing rotation of said control knob.

22. The apparatus as defined in claim 21 in which said control knob is provided with a plurality of circumferentially-spaced-apart cavities and in which said locking means comprises an outwardly extending finger portion receivable within a selected one of said circumferentially-spaced-apart cavities.

23. The apparatus as defined in claim 22 in which said control knob is provided with flow rate indicia for indicating fluid flow rate toward said fluid delivery means.
